(19) United States
(12) Patent Application Publication
(10) Pub. No.: US 2014/0348873 A1
(43) Pub. Date: Nov. 27, 2014

(54) Urea-Silicone Gel for Hyperkeratosis Treatment

(71) Applicant: Fabiana Campanati Banov, Sugar Land, TX (US)

(72) Inventor: Fabiana Campanati Banov, Sugar Land, TX (US)

(73) Assignee: PROFESSIONAL COMPOUNDING CENTERS OF AMERICA, Houston, TX (US)

(21) Appl. No.: 13/900,375

(22) Filed: May 22, 2013

(51) Int. Cl. A61K 8/898; A61Q 19/00; A61K 8/97; A61K 8/73; A61K 8/31; A61K 8/42; A61K 8/92; A61K 8/49

(52) U.S. Cl. CPC A61K 8/898; A61K 8/922; A61Q 19/007; A61K 8/4913; A61K 8/735; A61K 8/31; A61K 8/42; A61K 8/498; A61K 8/97; A61K 8/975
USPC 424/195.17; 514/63; 424/725; 514/54; 424/764; 424/777; 424/735; 424/744

(57) ABSTRACT

Compositions and methods for urea silicone gels for treating hyperkeratotic skin diseases that may benefit from barrier protection and from urea silicone gel's ability to return water balance to the skin are disclosed. Skin conditions that may be treated with urea silicone gels may be excessive dryness, callus, corns, old and new scars, and keloids, among others. Disclosed urea silicone gels may include micronized urea USP, and an anhydrous silicone base, among other ingredients. Anhydrous silicone base may include Amazonian oils such as pracaxi oil and seje oil, which are rich in oleic, linolenic, linoleic acids, and sterols, particularly beta-sitosterol and stigmasterol, which may increase skin permeability to urea. Additionally, because of the high content of unsaturated fatty acids of pracaxi oil and seje oil, disclosed urea silicone gel may exhibit an enhanced hydrating effect on skin cells, over-moisturizing and hydrating tough, thick hyperkeratotic conditions.

### CROSS-REFERENCE TO RELATED APPLICATIONS

The present application is related to U.S. patent application Ser. No. 13/900,360, filed on even date herewith and having an Attorney Docket No. of 61574-820968, and entitled “Urea Silicone Gel Scars and Hydration Treatment.”

### BACKGROUND

1. Field of the Disclosure

The present disclosure relates to topical delivery of urea, and more specifically to compositions and methods for hyperkeratosis treatment.

2. Background Information

Hard skin typically occurs on hands, feet, elbows, or other areas prone to friction, dryness and irritation. These areas of hard skin may be painless, as is usually the case with calluses, or they may be painful and inflamed. You can reduce or remove hard skin by avoiding sources of pressure and friction, removing areas of thickened skin and applying medicated creams and moisturizers to those areas.

Among all the parts of the body that are subjected to constant pressure and friction throughout the day, the feet bear most of the brunt. This causes the layer of the skin to become thick and hard, resulting in a condition known as foot callus. This is one of the most common foot problems in people in most parts of the world. Calluses do not sweat, therefore making the skin of the feet drier. Therefore, what is required, is a treatment that would cause the calluses to break down in order to reduce the dryness and cracking of the feet.

Moreover, keloids are fibrous overgrowths induced by cutaneous injury clinically, keloids behave like benign dermal fibro-proliferative tumors as they continue to grow and extend beyond the confines of the original wound margins, without evidence of spontaneous regression as observed in hypertrophic scars. Histologically, keloids and hypertrophic scars differ from normal skin and normal scars by their rich vasculature, a high density of mesenchymal cells, a thickened epidermal cell layer, an increased infiltration of inflammatory cells including lymphocytes, mast cells (MCs), and macrophages, and an abundant deposition of extracellular matrix (ECM). Abnormality in collagen synthesis, leading to an imbalance in ECM metabolism, has been recognized as an essential factor in the pathogenesis of several fibrotic diseases, including keloids.

Creams for calluses treatment generally include salicylic acid, which exhibit side effects such as skin irritation, dryness, peeling, and redness. Skin irritation may occur in the form of itchiness, burning, or stinging. The treated area may become red, swollen, or may develop skin rash.

Urea topical compositions do not exhibit side effects described in the previous paragraph because of urea's special ability to hydrate dry skin by drawing moisture into the cell structure of the stratum corneum. Urea works by breaking down hard, dead skin cells. Urea may also help the skin draw in and retain moisture to treat hard skin resulting from dryness. Urea breaks down skin cells associated with calluses and speeds the overall process of removal and healing. Urea softens hard skin because of its special ability to hydrate dry skin by drawing moisture into the cell structure of the stratum corneum.

There is therefore a need for compositions and methods for urea topical gels for enhanced treatments for hyperkeratotic skin diseases, which may allow topical delivery of urea with increased skin permeability, presenting reduced side effects.

### SUMMARY

Compositions and methods for urea silicone gel are described. Disclosed urea silicone gel may include at least two Amazonian oils such as pracaxi oil and seje oil that may enhance skin permeability to urea. In one embodiment, urea silicone gel may include micronized urea USP as active pharmaceutical ingredient (API), and an anhydrous silicone base that includes Amazonian oils, among other ingredients.

Anhydrous silicone base within urea silicone gel may include pracaxi oil and seje oil, which are rich in oleic, linolenic, linoleic acids, and sterols, particularly beta-sitosterol and stigmasterol that may increase skin permeability to urea or any other API. In other embodiments, urea silicone gel may also include Plukenetia Volubilis seed oil and Inaja oil, among other oils.

In other embodiments, urea silicone gel may include a second with or without a third suitable API. Various suitable additives, known to those skilled in the art, may be included in disclosed urea silicone gel.

Suitable amount of micronized urea USP within urea silicone gel may be from about 15% by weight to about 40% by weight; most suitable amount may be from about 15% by weight to about 25% by weight. Amount of anhydrous silicone base that may be included in disclosed urea silicone gel may range from about 5% by weight to about 85% by weight; most suitable amount may be of about 5% by weight to about 60% by weight.

In one embodiment, producing urea silicone gel may be achieved by mixing urea silicone gel's components in a homogenizer, such as a high speed shear homogenizer. The method may further include dispersing urea silicone gel in a vessel employing a high speed shear homogenizer, dispersing urea silicone gel at speeds of about 5,000 rotations per minute (RPM).

Disclosed urea silicone gel may be applied on body surface in order to moisturize skin and treat hyperkeratotic skin diseases such as callus, keloids, and corns, among others. Urea silicone gel may dissolve the intercellular matrix of the cells of the stratum corneum, promoting desquamation of scaly skin, eventually resulting in softening of hyperkeratotic areas. Additionally, urea silicone gel helps the stratum corneum maintain its capacity to retain water, effectively stimulating skin hydration and providing long-term results. Due to Amazonian oils within urea silicone gel, disclosed urea silicone gel may also exhibit anti-inflammatories and antimicrobial properties.

Furthermore, silicone within anhydrous silicone base increases hydration of stratum corneum and therefore facilitates regulation of fibroblast production and reduction in collagen production, resulting in softer and flatter scars. Additionally, silicone may protect the scarred tissue from bacterial invasion and may prevent bacteria-induced excessive collagen production in the scar tissue.

In other embodiments, disclosed urea silicone gel may be employed to treat skin spots, scars, and stretch marks, among other skin conditions

In one embodiment, urea silicone gel may be applied to any area of skin such as, hands, feet, elbows, joints, and other parts of the skin.

According to an embodiment, urea silicone gel may be applied directly to the affected area such as scar, callus, corn, or keloid, among others; twice daily (about 2 to 6 grams), during about 10 to 14 weeks.

Increasing the permeability to urea, the time of treatment may be significantly reduced, therefore reducing the time of results of treatment to a period of from about 2 weeks to about 8 weeks. Urea silicone gel effectively stimulates skin hydration and may provide long-term results.

Numerous other aspects, features of the present disclosure may be made apparent from the following detailed description.

### DETAILED DESCRIPTION

The present disclosure is here described in detail. Other embodiments may be used and/or other changes may be made without departing from the spirit or scope of the present disclosure. The illustrative embodiments described in the detailed description are not meant to be limiting of the subject matter presented here.

### DEFINITIONS

As used here, the following terms have the following definitions:

“Treating” and “Treatment” refer to reduction in severity and/or frequency of symptoms, elimination of symptoms and/or underlying cause, prevention of the occurrence of symptoms and/or their underlying cause, and improvement or remediation of damage.

“Permeation enhancement” refers to an increase in the permeability of the skin to the selected active pharmaceutical ingredient.

“Emollient” refers to a substance having the quality of softening or soothing the skin.

“Gel” refers to a colloid in which the disperse phase has combined with the dispersion medium to produce a semisolid material, such as a jelly that may include a cosmetic, medicinal, or other preparation.

“Scar” refers to a growth of collagen beneath the skin that may be formed as the result of wound healing.

“Keloids” refers to benign fibrous growths that occur after trauma or wounding of the skin and present a major therapeutic dilemma to the dermatologist because of frequent recurrences.

“Hyperkeratosis” refers to the thickening of the stratum corneum, often associated with a qualitative abnormality of the keratin, and also usually accompanied by an increase in the granular layer.

“Callus” refers to toughened area of skin which has become relatively thick and hard in response to repeated friction, pressure, or other irritation.

“Corn” refers to a specially shaped callus of dead skin that usually occurs on thin or glabrous skin surfaces.

“Keratin” refers to a fibrous scleroprotein that occurs in the outer layer of the skin.

“Silicone” refers to polymeric organic silicon compounds obtained as oils.

### DESCRIPTION

Embodiments of the present disclosure may be directed towards compositions and methods for urea silicone gels that include Amazonian oils, which may enhance skin permeability to urea or any other active pharmaceutical ingredient (API). Urea silicone gels may be applied on body surface in order to moisturize skin and treat hyperkeratotic skin diseases such as callus, keloids, and corns, among others.

Composition

In one embodiment, the present disclosure provides topical formulations that may be employed for hyperkeratotic conditions.

Disclosed urea silicone gel may include micronized urea USP, and an anhydrous silicone base, among other ingredients.

Active Pharmaceutical Ingredients

Urea, CO(NH₂)₂, is found in the epidermis; moreover, urea may be synthesized in the laboratory. Urea plays a vital role in maintaining the skin's moisture balance and the suppleness of the skin. Urea is known to be a debriding agent; meaning urea may help in getting rid of the dead, damaged, or infected tissues. When urea-including compositions, such as urea silicone gels, are applied to the callused feet, urea binds with the keratin to break keratin down thus, helping in loosening the hard and scaly skin. Moreover, because urea has a high adherence towards water, urea may draw moisture into the skin, such as in feet, therefore making the skin soft and healing the cracks and fissures. Because of urea's super-hydrating effect on skin cells, urea over-moisturizes and hydrates tough, thick callus.

Additionally, urea is non-toxic, non-allergenic, colorless, and odorless. Furthermore, urea may have anti-fungal and anti-microbial properties that may promote fast healing of dry cracked split skins and other types of skin problems.

Urea is naturally present in healthy skin, but when the skin is dry, and in some skin conditions the level of urea in the skin may be reduced. In the epidermis of healthy skin there is approximately 28 micrograms of urea per 2.5 square centimeters. In dry skin urea concentration may be diminished by about 50%. Urea can generally increase water content to the skin to a level of 97.8%.

Amount of micronized urea USP in disclosed urea silicone gel may be of about 15% by weight to about 40% by weight. Most suitable amount of micronized urea USP may be from about 15% by weight to about 25% by weight; depending on the skin condition to be treated.

In other embodiments, urea silicone gel may include a second with or without a third API to provide urea silicone gel with additional usage benefits. The second and third APIs may be a suitable pharmaceutical agent, herbal extract, and/or cosmetic agent, such as hydroxy-acids, among others.

According to an embodiment, urea may be combined with anhydrous silicone base in order to fabricate urea silicone gels. Anhydrous silicone base may include unique ingredients that may provide urea silicone gel potential healing and soothing power, emolliency and enhanced skin penetration.

Pracaxi Oil

In one embodiment, disclosed urea silicone gel include a natural oil from the Amazon forest, named pracaxi oil which exhibits moisturizing properties, as well as antimicrobial properties. Additionally, pracaxi oil may enhance the penetration of urea in skin, allowing a better absorption of micronized urea within urea silicone gel.

Pracaxi oil may be obtained from the seed oil of Pentaclethara Macroloba tree. Pracaxi oil may include about 20% of behenic acid and about 35% of oleic acid. In some cases, pracaxi oil may include more than these percentages. Behenic acid, oleic acid, and lauric acid, when used by themselves, may be irritating when applied to the skin. While having an irritating effect on the skin, behenic acid, oleic acid, and lauric acid, are also effective vehicles at delivering drugs, such as urea, through the skin. As the behenic acid and oleic acid are present within pracaxi oil, the effects of the acids may be less irritating on the skin, and as such makes pracaxi oil a good skin permeation enhancer. Pracaxi oil has been widely employed for its cosmetic, therapeutic, and medicinal properties. Pracaxi oil is rich in organic acids with antioxidant, antibacterial, antiviral, antiseptic, antifungal, anti-parasitic, and anti-hemorrhagic properties.

Pracaxi oil may have a high amount of solid matter, not fatty acids, which make pracaxi oil solidify in cooler temperatures. The solid matter has gentle moisturizers and high cellular renewal properties, includes vitamin E and has essential fatty acids which make pracaxi oil suitable oil for hyperkeratosis treatment. Additionally, pracaxi oil may improve the appearance of stretch marks.

The fatty acid composition of pracaxi oil is illustrated below in Table 1.

TABLE 1
Fatty acid composition of pracaxi oil.

| Fatty Acidds | Carbon Atoms | Composition % |
|---|---|---|
| Lauric | 12:00 | 1.3000 |
| Myristic | 14:00 | 1.2100 |
| Palmitic | 16:00 | 2.0400 |
| Stearic | 18:00 | 2.1400 |
| Oleic | 18:10 | 44.3200 |
| Linoleic | 18:20 | 1.9600 |
| Linolenic | 18:30 | 2.3000 |
| Behenic | 22:00 | 19.6700 |
| Lignoceric | 24:00 | 14.8100 |

Amount of pracaxi oil within anhydrous silicone base may range from about 0.5% by weight to about 50% by weight; most suitable amount may be of about 1% by weight to about 10% by weight.

Seje Oil

Seje oil may be extracted from the mesocarp of the patauá palm and generally appears as a greenish-yellow and transparent liquid, with little odor and taste, having the physical appearance and composition of fatty acids that are similar to olive oil (Olea europaea). Seje oil has high content of unsaturated fatty acids. Seje oil has a high content of oleic acid therefore seje oil may be used as skin moisturizers. The dry mesocarp of patauá palm may include about 7.4% protein and possesses an excellent amino acid composition. Seje oil also includes α-tocopherol in its composition.

The fatty acid composition of seje oil is illustrated below in Table 2.

TABLE 2
Fatty acid composition of seje oil.

| Fatty Acids | Carbon Atoms | Composition % |
|---|---|---|
| Palmitic | 16:00 | 13.2 |
| Palmitoleic | 16:10 | — |
| Stearic | 18:00 | 3.6 |
| Oleic | 18:10 | 77.7 |
| Linoleic | 18:20 | 2.7 |
| Linolenic | 18:30 | 0.6 |
| Arachidic | 20:00 | 2 |
| Unsaturated | | 81.6 |

Amount of seje oil within anhydrous silicone base may range from about 1% by weight to about 50% by weight; most suitable amount may be of about 1% by weight to about 10% by weight.

In further embodiments other oils such as Plukenetia Volubilis seed oil, Inaja oil, Buriti, Tucuma, Bacuri, Ucuuba, Muru-Muru, and Copaiba, may be included in anhydrous silicone base within urea silicone gel.

Silicone

Anhydrous silicone base may include long chain silicone polymer (polysiloxanes), and silicone dioxide. Long chain silicone polymers cross link with silicone dioxide.

Silicone increases hydration of stratum corneum and therefore facilitates regulation of fibroblast production and reduction in collagen production resulting in softer and flatter scar. Additionally, silicone within anhydrous silicone gel may protect the scarred tissue from bacterial invasion and may prevent bacteria-induced excessive collagen production in the scar tissue.

Furthermore, anhydrous silicone base may modulate the expression of growth factors, fibroblast growth factor β (FGF β) and tumor growth factor β (TGF β). TGF β stimulates fibroblasts to synthesize collagen and fibronectin. FGF β normalizes the collagen synthesis in an abnormal scar and increases the level of collagenases, which breaks down the excess collagen, therefore the balance of fibrogenesis and fibrolysis is ultimately restored.

Amount of silicone within anhydrous silicone base may range from about 5% by weight to about 85% by weight; most suitable amount may be of about 5% by weight to about 60% by weight.

In further embodiments, various additives, known to those skilled in the art, may be included in urea silicone gel to facilitate the preparation of suitable forms for patient's application. For example additives may include humectants, pH adjusting agents, preservatives, antioxidants, silk amino acids, enzymes, treintoin, vitamins, fragrance, colorant, gelling agents, stabilizers, and surfactants, among others.

Methods of Elaboration

Various methods may be used to produce disclosed urea silicone gel. In one embodiment, an electronic mixing system may be used employing a high shear force. Urea silicone gel may be mixed through number of different speed settings and time to achieve the desired particle size.

In one embodiment, in order to make urea silicone gel, anhydrous silicone base may be processed through an ointment mill to provide trituration, dispersion, and reduce particle size of urea silicone gel. Anhydrous silicone base may be stirred under low shear conditions until a uniform formulation may be obtained. The urea silicone gel may be packed in suitable bottles or any suitable packaging.

Urea-Silicone Gel Therapeutically Use and Application

Urea silicone gel may cause hard dry skin cells to “unpack” and expose their water binding sites, therefore enabling the cell to absorb and retain additional moisture. This action is also known as hydrotopic solubilization.

In an embodiment, the use of the combination of pracaxi oil and seje oil within urea silicone gel, may increase the skin permeability to urea, passing the stratum corneum and reaching the target area, particularly, because of the oils' high concentrations of oleic, linolenic, linoleic acids and sterols, particularly beta-sitosterol and stigmasterol.

In one embodiment urea silicone gel may be applied on skin to soften and fade callus, corns, keloids, and scar tissues.

In another embodiment, urea silicone gel may be applied on skin to treat excessive dryness that may be produced by pollutants or household and industrial chemicals.

Increasing the permeability to urea, the time of treatment may be significantly reduced, therefore reducing the time of results of treatment to a period of from about 1 week to about 4 weeks.

In one embodiment, urea silicone gel may be applied to any area of skin such as hands, feet, elbows, and joints, among others.

According to an embodiment, urea silicone gel may be applied directly to skin area to be treated, twice daily (about 2 to 6 grams), during about 10 to about 14 weeks.

In some embodiments, patients may soak the affected area in warm water for about 5 minutes prior to applying disclosed urea silicone gel in order to help soften the skin.

In one aspect of the present disclosure, urea silicone gel may be administered without being covered by an adhesive bandage, patch or other physical barrier bonded to the administration area. In other embodiments, urea silicone gel may be administered being covered with an adhesive bandage, patch or other physical barrier bonded to the administration area.

Urea silicone gel may become touch dry within about one to three minutes of application to body surface.

### EXAMPLES

Example #1 is an embodiment of formulation of urea silicone gels which includes natural oils, such as Aloe Vera oil. Composition for example #1 urea silicone gel is described in table 3.

TABLE 3
Example #1 composition.

| Ingredients | Percentages |
|---|---|
| Aloe Vera Oil | 5-10% |
| Urea Silicone Gel | qs 100 g |

Example #2 is an embodiment of formulation of urea silicone gels which includes natural oils, such as Almond oil. Composition for example #2 urea silicone gel is described in table 4.

TABLE 4
Example #2 composition.

| Ingredients | Percentages |
|---|---|
| Almond oil | 5-10% |
| Urea Silicone Gel | qs 100 g |

Example #3 is an embodiment of formulation of urea silicone gels which includes natural oils, such as Avocado oil. Composition for example #3 urea silicone gel is described in table 5.

TABLE 5
Example #3 composition.

| Ingredients | Percentages |
|---|---|
| Avocado Oil | 5-10% |
| Urea Silicone Gel | qs 100 g |

Example #4 is an embodiment of formulation of urea silicone gels which includes natural oils, such as Wheat Germ oil. Composition for example #4 urea silicone gel is described in table 6.

TABLE 6
Example #4 composition.

| Ingredients | Percentages |
|---|---|
| Wheat Germ Oil | 5-10% |
| Urea Silicone Gel | qs 100 g |

Example #5 is an embodiment of formulation of urea silicone gels which includes moisturizers, such as Sodium Pyrrolidone Carboxylate. Composition for example #5 urea silicone gel is described in table 7.

TABLE 7
Example #5 composition.

| Ingredients | Percentages |
|---|---|
| Sodium Pyrrolidone Carboxylate | 1-3% |
| Urea Silicone Gel | qs 100 g |

Example #6 is an embodiment of formulation of urea silicone gels which includes moisturizers, such as Sodium Hyalutonate. Composition for example #6 urea silicone gel is described in table 8.

TABLE 8
Example #6 composition.

| Ingredients | Percentages |
|---|---|
| Sodium Hyaluronate 1% Solution | 1-5% |
| Urea Silicone Gel | qs 100 g |

Example #7 is an embodiment of formulation of urea silicone gels which includes moisturizers, such as Squalane. Composition for example #7 urea silicone gel is described in table 9.

TABLE 9
Example #7 composition.

| Ingredients | Percentages |
|---|---|
| Squalane | 2% |
| Urea Silicone Gel | qs 100 g |

Example #8 is an embodiment of formulation of urea silicone gels which includes moisturizers, such as Ceramide III. Composition for example #8 urea silicone gel is described in table 10.

TABLE 10
Example #8 composition.

| Ingredients | Percentages |
|---|---|
| Ceramide III | 0.05-0.1% |
| Urea Silicone Gel | qs 100 g |

Example #9 is an embodiment of formulation of urea silicone gels which includes vitamins, such as Vitamin E. Composition for example #9 urea silicone gel is described in table 11.

TABLE 11
Example #9 composition.

| Ingredients | Percentages |
|---|---|
| Vitamin E (liquid) | 0.1-1% |
| Urea Silicone Gel | qs 100 g |

Example #10 is an embodiment of formulation of urea silicone gels which includes natural extracts, such as Marigold Extract (Calendula). Composition for example #10 urea silicone gel is described in table 12.

TABLE 12
Example #10 composition.

| Ingredients | Percentages |
|---|---|
| Marigold Extract (Calendula) | 1-5% |
| Urea Silicone Gel | qs 100 g |

Example #11 is an embodiment of formulation of urea silicone gels which includes natural extracts, such as Peucedanum Ostrithium Liquid Extract (Masterwort). Composition for example #11 urea silicone gel is described in table 13.

TABLE 13
Example #11 composition.

| Ingredients | Percentages |
|---|---|
| Peucedanum Ostrithium Liquid Extract (Masterwort) | 1-3% |
| Urea Silicone Gel | qs 100 g |

Example #12 is an embodiment of formulation of urea silicone gels which includes natural extracts, such as Algae Extract. Composition for example #12 urea silicone gel is described in table 14.

TABLE 14
Example #12 composition.

| Ingredients | Percentages |
|---|---|
| Algae Extract | 1-10% |
| Urea Silicone Gel | qs 100 g |

Example #13 is an embodiment of formulation of urea silicone gels which includes natural extracts, such as Arnica Flower Tincture. Composition for example #13 urea silicone gel is described in table 15.

TABLE 15
Example #13 composition.

| Ingredients | Percentages |
|---|---|
| Arnica Flower Tincture | 1-3% |
| Urea Silicone Gel | qs 100 g |

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A method of treating hyperkeratosis, comprising applying to the skin an effective amount of a pharmaceutical composition that comprises a urea silicon gel and at least one Amazonian oil.

2. The method according to claim 1, wherein at least one Amazonian oil is selected from the group consisting of pracaxi oil, seje oil, plukenetia volubilis oil, and inaja oil.

3. The method according to claim 1, wherein the urea silicon gel further comprises micronized urea.

4. The method according to claim 3, wherein the pharmaceutical composition comprises from about 15% to about 40% micronized urea by weight.

5. The method according to claim 3, wherein the pharmaceutical composition from about 15% to about 25% micronized urea by weight.

6. The method according to claim 1, wherein the pharmaceutical composition further comprises anhydrous silicon.

7. The method according to claim 6, wherein the pharmaceutical composition comprises from about 5% to about 85% anhydrous silicon by weight.

8. The method according to claim 6, wherein the pharmaceutical composition from about 5% to about 60% anhydrous silicon by weight.

9. The method according to claim 1, wherein an effective amount of the pharmaceutical composition is about 2 to about 6 grams.

10. The method according to claim 1, wherein an effective amount of the pharmaceutical composition is applied to the skin for about 10 to about 14 weeks.

11. A pharmaceutical composition comprising aloe vera oil and urea silicone gel.

12. The composition of claim 11, wherein the pharmaceutical composition comprises about 5% to about 10% aloe vera oil by weight.

13. A pharmaceutical composition comprising almond oil and urea silicone gel.

14. The composition of claim 13, wherein the pharmaceutical composition comprises about 5% to about 10% almond oil by weight.

15. A pharmaceutical composition comprising avocado oil and urea silicone gel.

16. The composition of claim 15, wherein the pharmaceutical composition comprises about 5% to about 10% avocado oil by weight.

17. A pharmaceutical composition comprising wheat germ oil and urea silicone gel.

18. The composition of claim 17, wherein the pharmaceutical composition comprises about 5% to about 10% wheat germ oil by weight.

19. A pharmaceutical composition comprising sodium pyrrolidone carboxylate and urea silicone gel.

20. The composition of claim 19, wherein the pharmaceutical composition comprises about 1% to about 3% sodium pyrrolidone carboxylate by weight.

21. A pharmaceutical composition comprising sodium hyalutonate and urea silicone gel.

21. The composition of claim 21, wherein the pharmaceutical composition comprises about 1% to about 5% sodium hyalutonate by weight.

22. The composition of claim 21, wherein the sodium hyalutonate is in 1% solution.

23. A pharmaceutical composition comprising squalane and urea silicone gel.

24. The composition of claim 23, wherein the pharmaceutical composition comprises about 2% squalane by weight.

25. A pharmaceutical composition comprising ceramide III and urea silicone gel.

26. The composition of claim 25, wherein the pharmaceutical composition comprises about 0.05% to about 0.1% ceramide III by weight.

27. A pharmaceutical composition comprising vitamin E and urea silicone gel.

28. The composition of claim 27, wherein the pharmaceutical composition comprises about 0.1% to about 1% vitamin E by weight.

29. A pharmaceutical composition comprising calendula and urea silicone gel.

30. The composition of claim 29, wherein the pharmaceutical composition comprises about 1% to about 5% calendula by weight.
